(19) United States
(12) Patent Application Publication
(10) Pub. No.: US 2025/0248844 A1
(43) Pub. Date: Aug. 7, 2025

(54) OPHTHALMIC SURGICAL PROBE

(71) Applicant: Alcon Inc., Fribourg (CH)

(72) Inventors: Wesley Hastings Elden, Southlake, TX (US); Paul R. Hallen, Colleyville, TX (US); Firas Rahhal, Tarzana, CA (US); Steven T. Charles, Memphis, TN (US)

(21) Appl. No.: 19/013,156

(22) Filed: Jan. 8, 2025

Related U.S. Application Data
(60) Provisional application No. 63/548,888, filed on Feb. 2, 2024.

(51) Int. Cl. A61F 9/007

(52) U.S. Cl. CPC A61F 9/00736

(57) ABSTRACT

Embodiments of the present disclosure generally relate to a surgical probe. In certain embodiments, the surgical probe includes a handpiece configured to be held by a user, a tube, and one additional component disposed in the tube. The tube includes a proximal end coupled to the handpiece and a distal end opposite the proximal end that includes a distal tip. The distal tip is beveled and includes an end face at least partially defining a port for aspiration. The surgical probe further comprises at least one functional component (e.g., a soft tip, a diathermy tip, or an optical fiber) disposed in the tube.

### INTRODUCTION

The vitreous body, often referred to as the vitreous humor or simply “the vitreous,” is a transparent, colorless, and gelatinous mass that fills the space between the lens and the retina of the eyeball. The vitreous makes up about 80% of the volume of the eyeball and helps maintain the round shape of the eye. Additionally, the vitreous assists in absorbing external mechanical shocks to the eye, provides nutrients to the lens, and supports the retina.

The vitreous is mostly comprised of water with trace amounts of collagen and hyaluronic acid, which provide the vitreous with its gelatinous structure. Over time, however, the vitreous liquefies and condenses (e.g., shrinks) due to age and normal wear and tear. Eventually, the vitreous cannot fill the volume of the eye's vitreous cavity, and so the vitreous separates from the retina, also known as “posterior vitreous detachment” or “PVD.” PVD is common for older adults, and can lead to more serious complications, such as retinal detachment, where the retina peels away from underlying layers of supporting tissues.

When treatment of PVD is necessary, ophthalmic surgeons typically utilize a vitrectomy probe to completely separate the detaching vitreous from the retina, cut the separated vitreous into smaller fragments, and then suction the fragmented vitreous out of the eye. To separate the vitreous from the retina, the cutter of the vitrectomy probe is deactivated, and the port of the probe is brought in close proximity to the detaching vitreous to “grab” the vitreous and peel it away. However, due to the design of traditional vitrectomy probes, and more particularly, the relatively small port sizes thereof, which diminish greatly with smaller probe gauges, it is extremely difficult to grab and peel the vitreous from the retina effectively without causing damage to the retina.

### SUMMARY

The present disclosure relates to microsurgical tools, and more specifically, to ophthalmic microsurgical devices and methods of use thereof.

In certain embodiments a surgical probe includes a handpiece configured to be held by a user, a tube, and one additional component disposed in the tube. The tube includes a proximal end coupled to the handpiece and a distal end opposite the proximal end that includes a distal tip. The distal tip is beveled and includes an end face at least partially defining a port for aspiration.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope and may admit to other equally effective embodiments.

FIG. 1A illustrates a conventional surgical probe during an ophthalmic surgical procedure to treat posterior vitreous detachments.

FIG. 1B illustrates an enlarged side view of the surgical probe in FIG. 1A.

FIG. 2A illustrates a side view of an exemplary surgical probe, in accordance with certain embodiments of the present disclosure.

FIG. 2B illustrates a side view of another exemplary surgical probe, in accordance with certain embodiments of the present disclosure.

FIGS. 3A-3C illustrate enlarged cross-sectional side views of exemplary configurations of probe tips for the probes in FIGS. 2A and 2B, in accordance with certain embodiments of the present disclosure.

FIG. 4 illustrates an enlarged cross-sectional side view of an exemplary configuration for a probe tip of the probes in FIGS. 2A and 2B, in accordance with certain embodiments of the present disclosure.

FIGS. 5A-5F illustrate enlarged perspective views of exemplary configurations of probe tips for the probes in FIGS. 2A and 2B, in accordance with certain embodiments of the present disclosure.

FIG. 6A illustrates a magnified perspective view of an exemplary textured surface for the probes in FIGS. 2A and 2B, in accordance with certain embodiments of the present disclosure.

FIG. 6B illustrates enlarged cross-sectional side view of the exemplary textured surface in FIG. 6A, in accordance with certain embodiments of the present disclosure.

FIG. 7A illustrates an enlarged cross-sectional side view of another exemplary configuration of a probe, in accordance with certain embodiments of the present disclosure.

FIG. 7B illustrates an enlarged cross-sectional side view of another exemplary configuration of a probe, in accordance with certain embodiments of the present disclosure.

FIG. 7C illustrates an enlarged cross-sectional side view of yet another exemplary configuration of a probe, in accordance with certain embodiments of the present disclosure.

FIGS. 8A-8C illustrate enlarged cross-sectional side views of exemplary configurations of a handpiece for extending/retracting an inner portion of a probe relative to a tube of the probe, in accordance with certain embodiments of the present disclosure.

FIGS. 9A-9C illustrate enlarged cross-sectional side views of exemplary configurations of a probe, in accordance with certain embodiments of the present disclosure.

FIGS. 10A-10C illustrate enlarged cross-sectional side views of exemplary configurations of a probe, in accordance with certain embodiments of the present disclosure.

FIGS. 11A-11C illustrate enlarged cross-sectional side views of exemplary configurations of a probe, in accordance with certain embodiments of the present disclosure.

FIGS. 12A-12C illustrate enlarged cross-sectional side views of exemplary configurations of a probe, in accordance with certain embodiments of the present disclosure.

FIG. 13 illustrates a surgical system, in accordance with certain embodiments of the present disclosure.

FIG. 14 illustrates subsystems of a console of the surgical system of FIG. 13, in accordance with certain embodiments of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end, segment, or portion of a component refers to the end, segment, or portion that is closer to a patient's target tissue during use thereof. On the other hand, a proximal end, segment, or portion of the component refers to the end, segment, or portion that is distanced further away from the patient's target tissue.

As used herein, the term “about” may refer to a +/−10% variation from the nominal value. It is to be understood that such a variation can be included in any value provided herein.

The present disclosure relates to microsurgical tools, and more specifically, to ophthalmic microsurgical probes for manipulation of ocular materials/tissues and methods of use thereof.

As described above, during certain procedures, ophthalmic surgeons may utilize vitrectomy probes to separate the vitreous from the retina (e.g., create a posterior vitreous detachment, or “PVD”), prior to cutting and suctioning the vitreous from the eye. While effective for cutting operations, vitrectomy probes may be ineffective for engaging and extruding tissues and other ocular materials, such as the vitreous, and may even facilitate unwanted damage to tissues (e.g., the retina) adjacent to an operation site. The devices described herein address the deficiencies of certain existing methods and designs described above, and further reduce the risk of unwanted damage to peripheral tissues, by providing probes designed for effective engagement of the vitreous and other materials. Such probes include beveled probe tips that increase the area of vacuum generation at a target site without increasing probe gauge, thus facilitating improved tissue engagement as compared to other devices of similar gauge. In certain embodiments, the probes described herein further include one or more texturized surfaces for improved “grabbing” and manipulation of target tissues, and/or one or more surfaces formed of polymeric materials to reduce unwanted damage and enhance the safety thereof. In certain embodiments, the probes described herein further include one or more additional components, such as a diathermy tip or an optical fiber, which may provide additional functionality to the probe. Examples of this additional functionality can include coagulation, cauterization, photocoagulation, illumination, manipulation, etc.

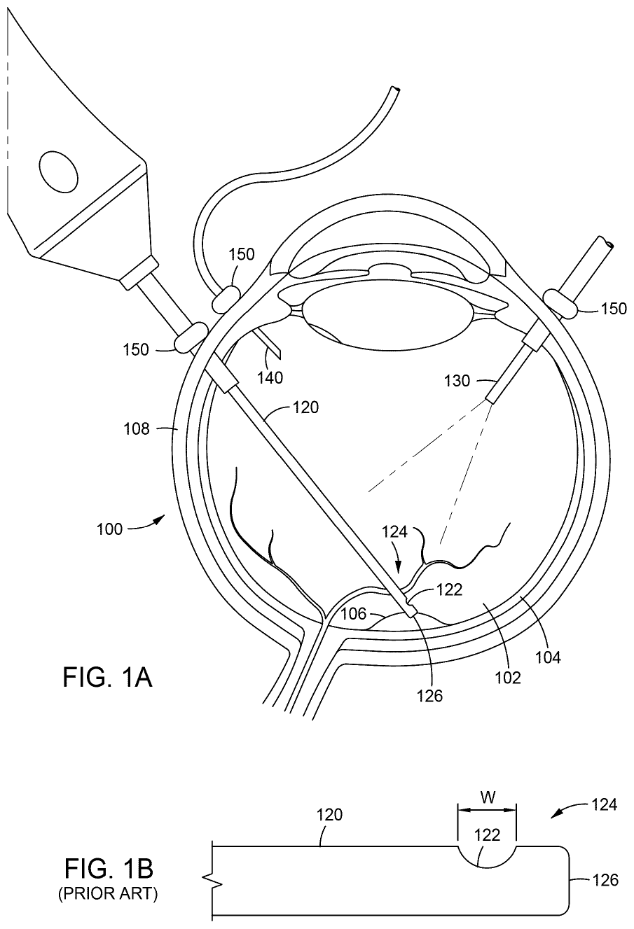

FIG. 1A illustrates a cross-sectional side view of exemplary eye 100 undergoing an ophthalmic procedure in which vitreous 102 is separated from retina 104 by conventional methods to form posterior vitreous detachment (PVD) 106 prior to cutting and suctioning vitreous 102 from eye 100. As shown in FIG. 1A, various microsurgical instruments are inserted into eye 100, including vitrectomy probe 120 for cutting and removing vitreous 102, endoilluminator 130 for providing illumination inside eye 100, and infusion cannula 140 for replacing fluid within eye 100 with saline solution and for maintaining intraocular pressure. Vitrectomy probe 120, endoilluminator 130, and infusion cannula 140 are typically inserted into eye 100 through respective trocar cannulas 150 that are inserted into incisions in sclera 108, as would be understood by skilled persons.

To separate vitreous 102 from retina 104 and create PVD 106, a surgeon may deactivate the cutter of vitrectomy probe 120 and bring port 122 at distal end 124 of the probe near the desired portion of vitreous 102 to “grab” the vitreous. Thereafter, vitrectomy probe 120 may be carefully pulled away from retina 104 to peel vitreous 102 from retina 104. However, due to port 122 being disposed through a sidewall of vitrectomy probe 120 rather than, e.g., distal end face 126, as well as the relatively small size of port 122 as a function of the gauge of vitrectomy probe 120, it may be extremely difficult to extrude vitreous 102 from retina 104 with vitrectomy probe 120 effectively without causing damage to retina 104.

FIG. 1B illustrates an enlarged side view of distal end 124 of vitrectomy probe 120 in FIG. 1A to better depict the arrangement of port 122. As shown, port 122 of vitrectomy probe 120 is disposed through a sidewall of the probe, rather than distal end face 126. Thus, when attempting to manipulate/extrude tissues or other materials within eye 100, e.g., vitreous 102, the surgeon must carefully position, rotate, and angle vitrectomy probe 120 such that port 122 is adjacent to a desired tissue or other material in order to “grab” the tissue or material. Not only that—the surgeon must also consider how to grab the tissue or other material while maintaining visualization of the tissue or material, without obstruction by probe 120. And, as previously mentioned, the relatively small size of port 122, represented as width W in FIG. 1B, may provide suboptimal vacuum generation for engagement with tissues or other materials, thereby further increasing the difficulty in manipulating tissues or other materials with vitrectomy probe 120.

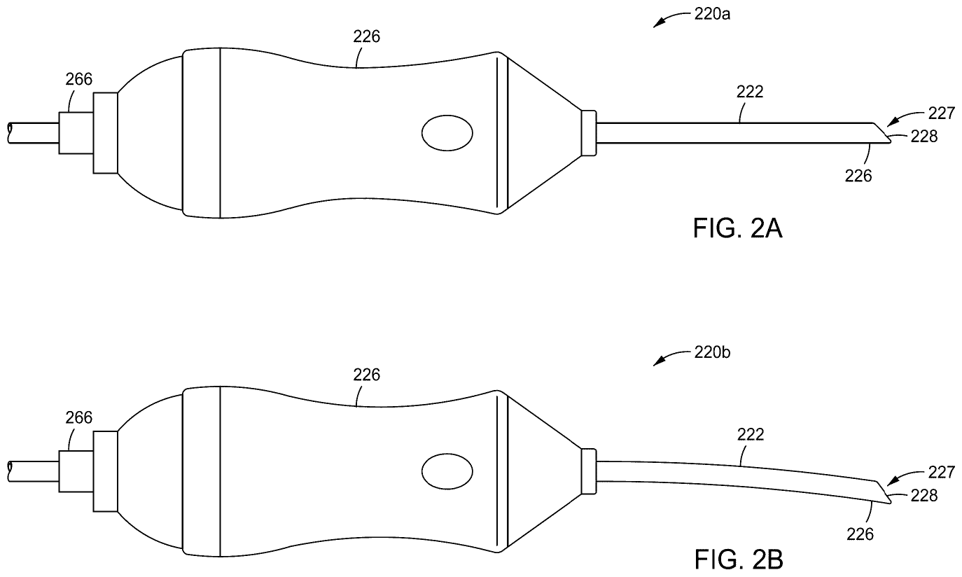

FIG. 2A illustrates a side view of an improved surgical probe 220a, in accordance with certain embodiments of the present disclosure. Probe 220a includes an elongated member that may be inserted into an eye, e.g., through a trocar cannula, for engaging and manipulating the vitreous and other tissues and/or materials. In certain embodiments, probe 220a is configured to create a posterior vitreous detachment, or PVD.

As shown, in certain embodiments, probe 220a comprises a hollow, cylindrical tube 222 (e.g., non-segmented) defining a longitudinal axis of probe 220a and having an outer diameter less than about 20 gauge. For example, in certain embodiments, tube 222 has a diameter of about 23 gauge, 25 gauge, 27 gauge, or less. In certain embodiments, tube 222 is segmented into two or more segments having outer diameters of different sizes. For example, in certain embodiments, a first proximal segment of tube 222 may have an outer diameter of about 23 or 25 gauge, while a second distal segment of tube 222 may have an outer diameter of about 25 or 27 gauge, respectively. In still other embodiments, however, probe 220a comprises a hollow triangular, quadrilateral, or polygonal tube having a plurality of longitudinal facets. Note that, as described herein, a distal segment, portion, or end of a component refers to the segment, portion, or end that is closer to a patient's target tissue during use thereof. On the other hand, a proximal segment, portion, or end of the component refers to the segment, portion, or end that is distanced further away from the patient's target tissue.

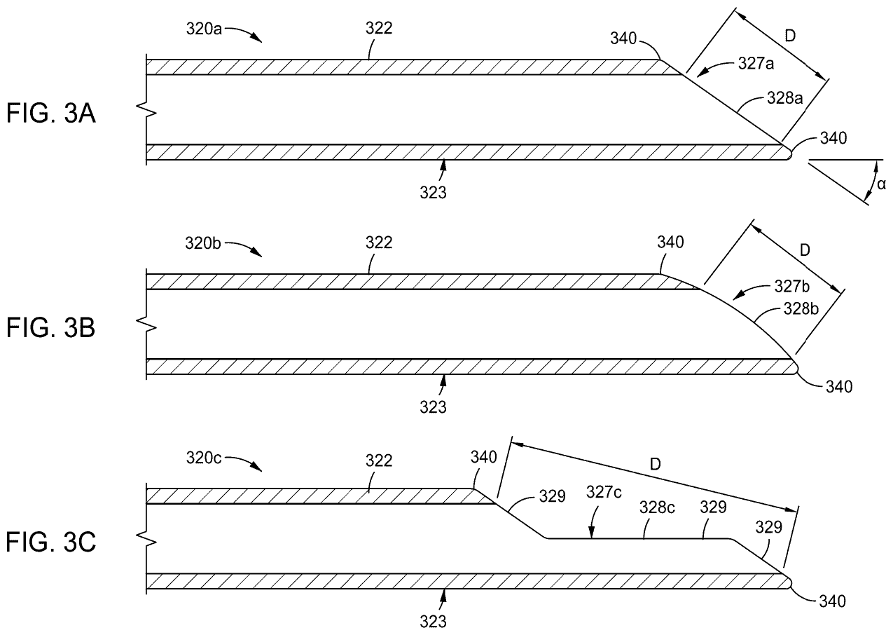

Tube 222 further comprises distal tip 226 at a distal end thereof. Distal tip 226 comprises end face 227 through which port 228 is disposed. Port 228, which is partially defined by end face 227, facilitates the provision of vacuum at a target tissue or material within a patient's eye for “grabbing” and manipulating the tissue or material during ophthalmic procedures. As shown, distal tip 226 is beveled (e.g., angled) at an angle that is non-normal relative to a major (longitudinal) axis 221 of probe 220a, thereby causing port 228 to have an elongated, e.g., ellipsoid shape. The beveled morphology of distal tip 226 and thus, the elongated shape of port 229, increases the surface area of vacuum generation at port 228 without requiring an increase in probe gauge. Accordingly, probe 220a enables improved suction or “purchase” of ocular tissues/materials and thus, easier manipulation thereof, at smaller probe gauges. For clarity, enlarged cross-sectional views of distal tip 226 and port 228 are illustrated in FIGS. 3A-3C, which are described in further detail below.

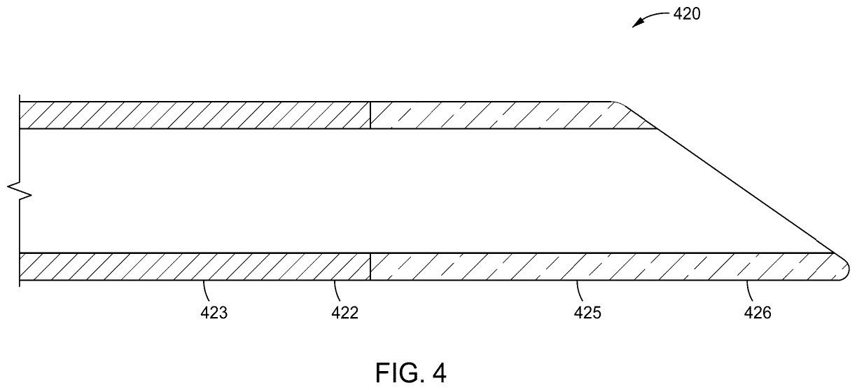

Tube 222 of probe 220a may be formed of any materials suitable for performing ophthalmic procedures. In certain embodiments, tube 222 comprises a plastic or polymeric material. In such embodiments, a portion or substantially all of tube 222 may be translucent or transparent. In certain other embodiments, tube 222 comprises more conventional surgical-grade materials, such as aluminum, stainless steel (e.g., 316 or 316L stainless steel), or other alloys. In particular examples, tube 222 is formed of Phynox, Elgiloy, or other suitable cobalt-chromium-nickel alloys. In particular examples, tube 222 is formed of nitinol or other suitable nickel-titanium alloys. In further embodiments, the tube 222 may comprise a combination of metallic and polymeric materials, as illustrated and described with reference to FIG. 4.

As further shown in FIG. 2A, a proximal end of tube 222 may, in certain embodiments, be partially and longitudinally disposed through a distal end of handpiece 260 and may be directly or indirectly attached thereto within an interior lumen of handpiece 260. In certain embodiments, handpiece 260 is a hand piece having an outer surface configured to be held by a user, such as a surgeon. For example, handpicce 260 may be ergonomically contoured to substantially fit the hand of the user. In certain embodiments, the outer surface may be textured or have one or more gripping features formed thereon, such as one or more grooves and/or ridges. Handpiece 260 may be made from any materials commonly used for such instruments and suitable for ophthalmic surgery. For example, handpiece 260 may be formed of a lightweight aluminum, a polymer, or other suitable material. In some embodiments, handpiece 260 may be sterilized and used in more than one surgical procedure or may be a single-use device. Handpiece 260 further includes one or more ports 266 at a proximal end thereof for providing ingress/egress for vacuum supply lines to be routed into an interior lumen of handpiece 260. For example, port 266 may provide a connection between handpiece 260 (and thus, probe 220a) and a vacuum supply line of a vacuum source within a surgical console.

FIG. 2B illustrates a side view of another exemplary surgical probe 220b, in accordance with certain embodiments of the present disclosure. Surgical probe 220b is substantially similar to surgical probe 220a, but for the presence of curvature in tube 222. Accordingly, tube 222 may be a curved cylindrical, triangular, quadrilateral, or polygonal tube. In certain embodiments, the tube curvature is formed at and/or adjacent to the distal end of tube 222, e.g., within 5-10 mm (millimeters) of the distal tip 226 of tube 222. Generally, the tube curvature may be shaped to match a curvature of the patient's eye, e.g., a retinal surface of eye 100 in FIG. 1A.

FIGS. 3A-3C illustrate enlarged cross-sectional side views of exemplary configurations of probes 320a, 320b, and 320c, which are representative of the surgical probes in FIGS. 2A and 2B, in accordance with certain embodiments of the present disclosure. More particularly, FIGS. 3A-3C depict distal tips 326a, 326b, and 326c of probes 320a, 320b, and 320c, respectively. Each distal tip 326a, 326b, and 326c includes a corresponding end face 327a, 327b, or 327c, respectively, through which a port 328a, 328b, or 328c, respectively, is disposed.

As shown in FIG. 3A, distal tip 326a of probe 320a is beveled such that end face 327a, which is substantially planar in this example, is disposed at a non-normal (non-perpendicular) angle α relative to a major axis 322 of probe 320a. Generally, the beveling of distal tip 326a creates a larger surface area, represented as dimension “D” in FIG. 3A, for port 328a, thus enabling greater vacuum generation thereat, for improved “grabbing” of ocular tissues and other materials during ophthalmic procedures at smaller probe gauges. Additionally, during many ophthalmic procedures requiring manipulation of tissues, the surgical probe is inserted into the eye through an incision and/or cannula disposed in the superotemporal quadrant of the eye, and so the distal tip of the surgical probe is brought toward a target tissue or material (e.g., the back surface of the vitreous) at an angle. Here, by beveling distal tip 326a of probe 320a such that end face 327a is disposed at an angle, probe 320a facilitates greater purchase (e.g., suction) of a target tissue or material, since end face 327a is configured to face the target tissue or material and create a “sealed” vacuum suction thereon. Accordingly, probe 320a facilitates improved manipulation of tissues and other ocular tissues as compared to conventional probes.

In certain embodiments, angle α is between about 0° and about 90° relative to the normal of major axis 322, such as between about 5° and about 70° relative to the normal of major axis 322, such as between about 10° and about 60° relative to the normal of major axis 322, such as between about 20° and about 40° relative to the normal of major axis 322, such as about 30° relative to the normal of major axis 322. In certain embodiments, angle α is between about 10° and about 30° relative to the normal of major axis 322, such as between about 15° and about 25° relative to the normal of major axis 322, such as about 18° relative to the normal of major axis 322.

As described above, end face 327a of probe 320a is substantially planar, and is connected to outer surface 323 by lateral edge 340, which may be rounded. However, other end face profiles/morphologies are also contemplated, such as those shown in FIGS. 3B and 3C. For example, in FIG. 3B, end face 327b comprises a curved or rounded profile. In certain embodiments, end face 327b comprises an outward curvature (e.g., convex), which may, in certain embodiments, match the curvature of a patient's eye, e.g., a retinal surface of eye 100. In certain other embodiments, end face 327b may comprise an inward curvature (e.g., concave). In the example of FIG. 3C, end face 327c comprises a staggered profile (e.g., a stepped profile) having a plurality of incremental, or stepped, segments 329. In such embodiments, segments 329 may be substantially planar as shown in FIG. 3C, or segments 329 may be curved or rounded. Note that although three segments 329 are depicted, more or less segments are also contemplated.

FIG. 4 illustrates an enlarged cross-sectional side view of an exemplary configuration of probe 420, which is representative of the surgical probes in FIGS. 2A and 2B, in accordance with certain embodiments of the present disclosure. As shown, probe 420 comprises tube 422 having portions thereof formed of at least two different materials. More particularly, tube 422 comprises a first, proximal portion 423 formed of a first material, and a second, distal portion 425 (which includes distal tip 426) formed of a second material. In certain embodiments, proximal portion 423 is formed of a surgical-grade metallic material, such as aluminum, stainless steel (e.g., 316 or 316L stainless steel), phynox, or another metal alloy (or other metal alloys), while distal portion 425 is formed of a plastic or polymeric material. In such embodiments, proximal portion 423 provides the necessary stiffness for maneuvering probe 420 within the intraocular space during an ophthalmic procedure, while distal portion 425, which may be contacted against various tissues in the eye, provides a degree of pliability to reduce the risk of damage to these tissues. Furthermore, utilization of a polymeric distal portion 425 may facilitate improved engagement with ocular tissue and other materials, as a softer probe tip is more likely to “grab” such tissue and materials. Accordingly, the exemplary probe 420 in FIG. 4 may facilitate improved safety and efficiency during certain ophthalmic procedures as compared to more conventional probes.

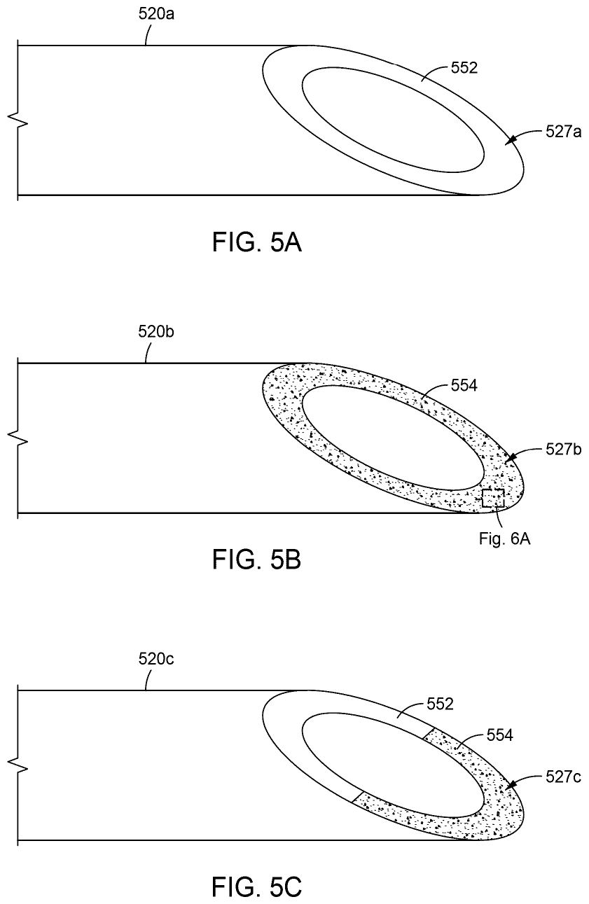

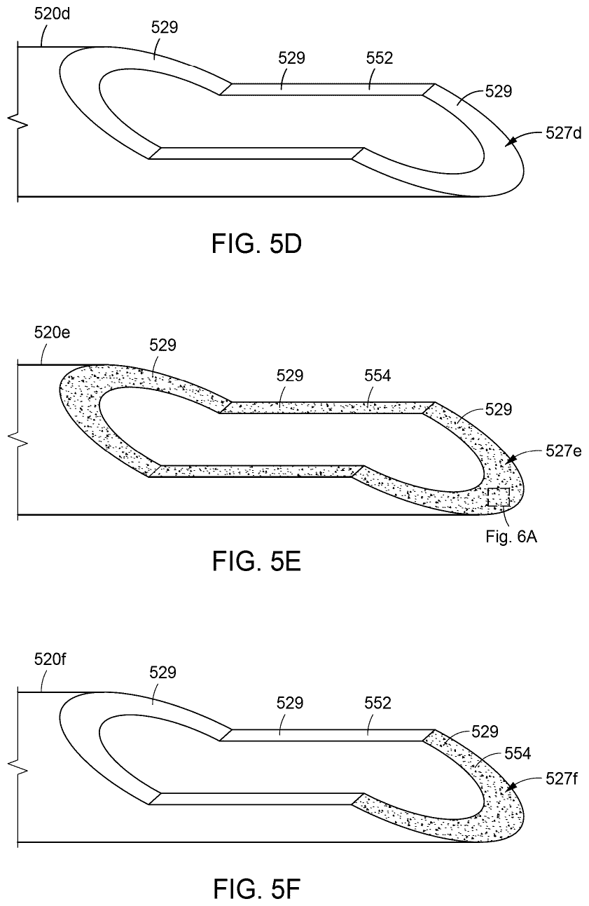

FIGS. 5A-5F illustrate enlarged perspective views of exemplary configurations of probes, which are representative of the surgical probes in FIGS. 2A and 2B, in accordance with certain embodiments of the present disclosure. More particularly, FIGS. 5A-5F illustrate end faces 527a, 527b, 527c, 527d, 527c, and 527f of probes 520a, 520b, 520c, 520d, 520c, and 520f, respectively. End faces 527a-527c are planar in profile, as described with reference to FIG. 3A above, while end faces 527d-527f are staggered, or stepped, as described with reference FIG. 3C above.

As shown, planar end face 527a comprises substantially smooth, or untextured, surface 552 across an entire surface area thereof. Conversely, planar end face 527b comprises textured surface 554 across an entire surface area thereof. Alternatively, planar end face 527b comprises both smooth surface 552 and textured surface 554 across different portions of a surface area thereof. Similarly, staggered end face 527d, which includes a plurality of segments 529, comprises smooth surface 554 across an entire surface area thereof. Meanwhile, staggered end face 527c comprises textured surface 554 across an entire surface area thereof, and staggered end face 527f comprises both smooth surface 552 and textured surface 554 across different portions of a surface area thereof (here, a distal segment 529 comprises textured surface 554 while the remainder comprise smooth surface 552, though other arrangements are also contemplated). In examples including textured surfaces 554, the textured surface may increase friction between the probe and the target tissue or other ocular material by providing a higher coefficient of friction, thereby improving engagement of the probe with such tissue or material. Furthermore, with a higher coefficient of friction, the normal force needed to engage the tissue or material with the probe is reduced. And, as a result of reducing the normal force applied to the probe during a given procedure, the risk of injury or indentation to the eye may be reduced.

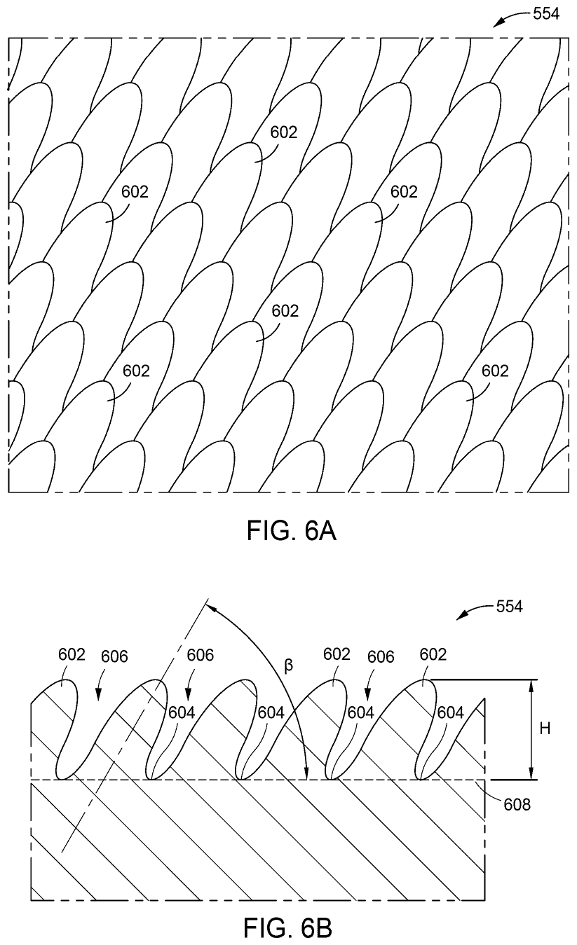

FIG. 6A illustrates an enlarged perspective view of textured surface 554 shown in FIGS. 5B, 5C, 5E, and 5F, in accordance with certain embodiments of the present disclosure. FIG. 6B illustrates a cross-sectional side view of textured surface 554, in accordance with certain embodiments of the present disclosure. For clarity, FIGS. 6A and 6B are herein described together.

As shown, textured surface 554 includes a plurality of raised surface features 602. Features 602 are configured to increase a coefficient of friction between a probe (e.g., probes 220a and 220b) and a target tissue or ocular material, thereby improving engagement between the probe and the target tissue or material during an ophthalmic procedure. In certain embodiments, features 602 comprise micro- or nano-posts. In certain embodiments, features 602 comprise micro- or nano-hooks. Features 602 may be arranged in any suitable arrangement on, e.g., an end face of a probe. For example, in certain embodiments, features 602 may be arranged in one or more linear arrays on an end face of a probe. In certain other embodiments, features 602 may be arranged in a circular or rotationally symmetric array on an end face of a probe. In the illustrated examples, features 602 may be formed by application of laser energy to the end face of a probe, e.g., probes 220a and 220b. In certain embodiments, a femtosecond or picosecond laser may be used.

Generally, features 602 have a height H that is measured from troughs 604 of traces 606 disposed between features 602, and that is further measured perpendicularly from effective surface 608 of textured surface 554, which is defined by a surface passing through troughs 604. In certain embodiments, the height H is between about 2 μm (micrometers) to about 10 μm, such as between about 3 μm and about 9 μm, such as between about 4 μm and about 8 μm, such as between about 5 μm and about 7 μm. In still other embodiments, the height H of features 602 may be greater than 10 μm or smaller than 2 μm. In further embodiments, the height H of features 602 may vary across textures surface 554.

In certain embodiments, features 602 are disposed at an angle β incident to effective surface 608. Angling of features 602 may facilitate “grabbing” of the target tissue/material when the probe is moved against the target tissue/material in one direction, and “release” or the target tissue/material when the probe is moved against the target tissue/material in a second, opposite direction. Such bidirectional functionality makes probe engagement with target tissues/material efficient and predictable. In certain embodiments, the angle β is within a range of 10° to 90°, where 90° is perpendicular to effective surface 608. In certain embodiments, the angle β may be within a range of about 20° to about 70°, about 20° to about 55°, about 30° to about 60°, about 40° to about 50°, about 20° to about 50°, or about 30° to about 45°.

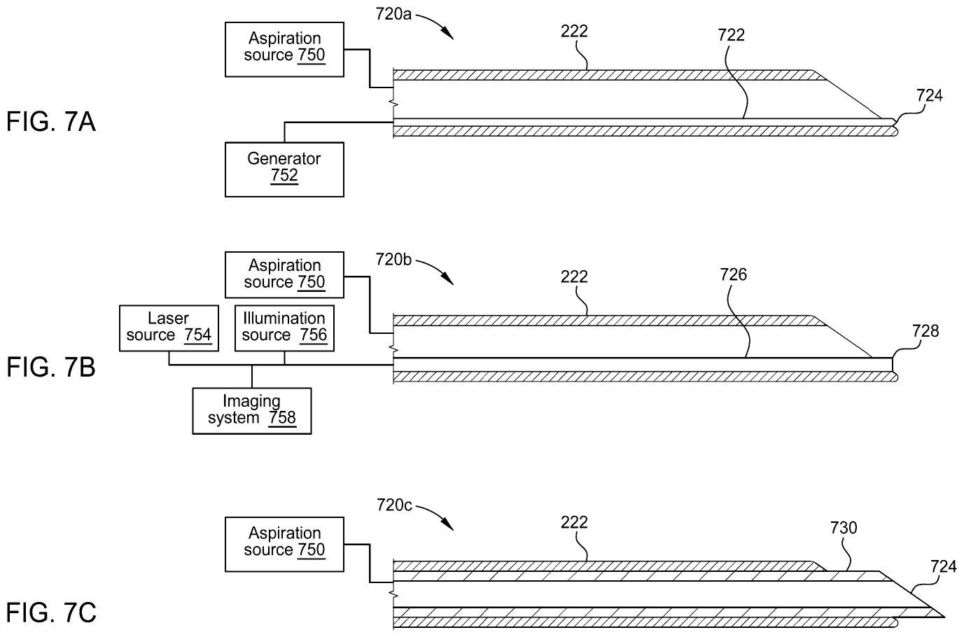

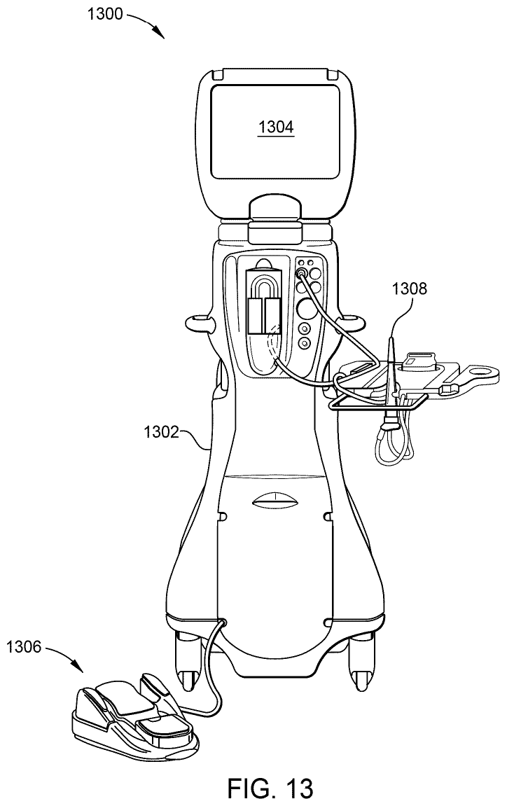

FIG. 7A illustrates an enlarged cross-sectional side view of an exemplary configuration of a probe 720a, in accordance with certain embodiments of the present disclosure. In some embodiments, probe 720a includes handpiece 260. Probe 720a is illustrated to be operably coupled to an aspiration source 750 and a generator 752. In certain embodiments, the aspiration source 750 and/or the generator 752 are included in probe 720a (e.g., included in handpiece 260). In certain other embodiments, the aspiration source 750 and/or the generator 752 are included in a console that is connected/attached to probe 720a (e.g., connected/attached to handpiece 260 via a cable or other tether) as described in greater detail with respect to FIG. 13.

In the illustrated example, probe 720a includes tube 222 and a functional component in the form of diathermy tip 722 having distal end 724, which can be used to coagulate tissue (e.g., stop bleeding) or cauterize tissue (e.g., prevent bleeding). Diathermy tip 722 is disposed in tube 222 such that distal end 724 is capable of contacting a target tissue. In some examples, diathermy tip 722 is disposed adjacent to a longest portion of tube 222, and diathermy tip 722 may be fixed within tube 222 by an adhesive, an epoxy, a weld, etc. Although examples are described in illustrated showing diathermy tip 722 disposed within tube 222, it is to be appreciated that, in some examples, diathermy tip 722 can be disposed outside of tube 222 such that diathermy tip 722 is fixed to an outer portion of tube 222. Diathermy tip 722 includes at least one electrically conductive material such as copper, aluminum, stainless steel, etc. In certain embodiments, a portion of diathermy tip 722 may be disposed within an electrical insulator material or a dielectric material configured to electrically isolate the portion of diathermy tip 722 from tube 222.

In one or more examples, diathermy tip 722 is usable for wet field bipolar diathermy, micro-diathermy, etc. In various embodiments, a proximal end of diathermy tip 722 is connected to the generator 752 which generates high frequency (e.g., radio frequency) electricity. The electricity is transmitted through diathermy tip 722 to distal end 724 and the electricity is converted into heat at distal end 724. While the generator 752 is energized, a surgeon briefly contacts tissue with distal end 724 to coagulate/cauterize a target tissue.

FIG. 7B illustrates an enlarged cross-sectional side view of an exemplary configuration of a probe 720b, in accordance with certain embodiments of the present disclosure. In one or more embodiments, probe 220b includes handpiece 260. Probe 720b is illustrated to be operably coupled to the aspiration source 750, a laser source 754, an illumination source 756, and an imaging system 758. In certain embodiments, the aspiration source 750, the laser source 754, the illumination source 756, and/or the imaging system 758 are included in probe 220b (e.g., included in handpiece 260). In certain other embodiments, the aspiration source 750, the laser source 754, the illumination source 756, and/or the imaging system 758 are included in a console that is connected/attached to probe 720b (e.g., connected/attached to handpiece 260 via a cable or other tether) as described in greater detail with respect to FIG. 13.

As shown in FIG. 7B, probe 720b includes tube 222 and a functional component in the form of at least one optical fiber 726. In certain embodiments, probe 720b may be an endoilluminator, a laser probe, an illuminated laser probe, an endoscopic camera, etc. Optical fiber 726 is configured to transmit/receive light, which can be light for illumination, light for photocoagulation or ablation (e.g., laser light), or captured light for endoscopic/imaging functionality. In the illustrated example, optical fiber 726 is disposed within tube 222. In certain embodiments, optical fiber 726 is arranged in tube 222 differently for different functionalities of optical fiber 726. For laser functionality via the laser source 754, optical fiber 726 is disposed in tube 222 and fixed within tube 222 such that distal end 728 is adjacent to an open end of tube 222. For illumination functionality via the illumination source 756, optical fiber 726 is disposed in tube 222 such that distal end 728 is generally adjacent to the open end of tube 222 (e.g., distal end 728 can be slightly recessed into tube 222 or distal end 728 may slightly extend out from tube 222). For imaging applications via the imaging system 758, optical fiber 726 may be disposed in tube 222 such that distal end 728 extends out from tube 222 if distal end 728 includes a lens, a camera, an image sensor, etc. For other imaging applications via the imaging system 758, optical fiber 726 can be disposed in tube 222 and fixed within tube 222 such that distal end 728 is adjacent to the open end of tube 222.

In various embodiments, a proximal end of optical fiber 726 is connected to the illumination source 756, such as a white light source (e.g., for illumination), the proximal end of optical fiber 726 is connected to the laser source 756 (e.g., for photocoagulation), or the proximal end of optical fiber 726 is connected to the imaging system 758 (e.g., for endoscopic imaging or image capture). For example, light enters the proximal end of optical fiber 726, and the light is transmitted out from distal end 728 of optical fiber 726 to illuminate a surgical site or photocoagulate/ablate a surgical target. In some examples, light enters distal end 728 of optical fiber 726, and the light is transmitted out from the proximal end of the optical fiber 726 to view a surgical target or capture images depicting the surgical target. In certain embodiments, the proximal end of the optical fiber 726 can be in communication with a camera, a lens, an image sensor, other computing device, etc.

In some embodiments, probe 720b includes multiple optical fibers 726. For example, probe 720b may include one optical fiber 726 for illumination and another optical fiber 726 for photocoagulation. In various examples, probe 720b can include one optical fiber 726 for illumination and an additional optical fiber 726 configured for endoscopic image capture. In examples in which probe 720b includes optical fiber 726 configured as an endoscopic camera, (e.g., distal end 728 may include a camera, a lens, an image sensor, etc.) and images captured by the endoscopic camera can be displayed in substantially real time on a monitor of a device in wired or wireless communication with the endoscopic camera. It is to be appreciated that probe 720b may include any number of optical fibers 726 which can be configured to provide different functionality or the same functionality. For example, probe 720b may be configured as a multi-spot laser probe such that probe 720b can include multiple optical fibers 726 configured to transmit laser light for photocoagulation.

FIG. 7C illustrates an enlarged cross-sectional side view of an exemplary configuration of a probe 720c, in accordance with certain embodiments of the present disclosure. In various embodiments, probe 220c includes handpiece 260. Probe 720c is illustrated to be operably coupled to the aspiration source 750. In some embodiments, the aspiration source 750 is included in probe 720c (e.g., included in handpiece 260). In other embodiments, the aspiration source 750 is included in a console that is connected/attached to probe 720c (e.g., connected/attached to handpiece 260 via a cable) as described in greater detail with respect to FIG. 13.

Probe 720c is illustrated to include tube 222 and a functional component in the form of soft tip 730. In various examples, soft tip 730 is manufactured from a material (e.g., silicone, rubber, a thermoplastic polymer, etc.) which is soft enough to manipulate retina 104, or other ocular tissues, without causing trauma or injury. In certain embodiments, soft tip 730 includes a hollow/tubular structure attached to an inner wall of tube 222 and extending from a distal end thereof. In such embodiments, soft tip 730 can extend an entire or partial length of tube 222. In certain embodiments, soft tip 730 is attached/adhered to only the distal end of tube 222 and has a similar lateral geometry such that together tube 222 and soft tip 730 form a continuous tubular shape.

In some examples, probe 720c is part of a backflush handpiece, which can provide active or passive aspiration through soft tip 730. In one or more embodiments, distal end 732 of soft tip 730 has the same geometry (e.g., tapers at the same angle) as end face 227. In certain embodiments, distal end 732 of soft tip 730 has a different geometry (e.g., tapers at a different angle) from end face 227. In some embodiments, distal end 732 of soft tip 730 has a geometry that is flat/normal, does not include a taper, includes multiple tapers, etc.

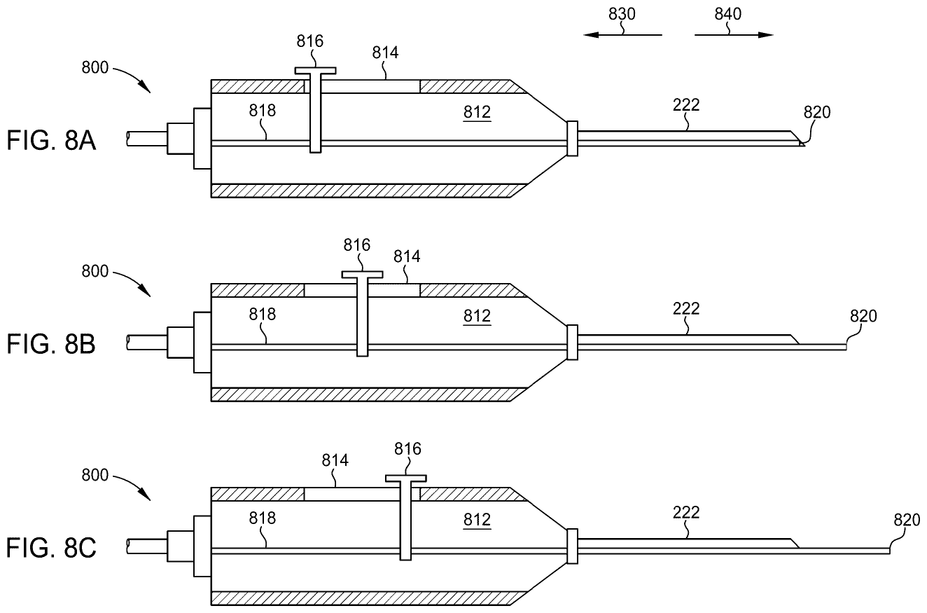

FIGS. 8A-8C illustrate enlarged cross-sectional side views of exemplary configurations of a handpiece 800 for extending/retracting a portion of a probe, e.g., an inner portion of the probe such as the additional functional components described with reference to FIGS. 7A-7C, relative to a tube of the probe, in accordance with certain embodiments of the present disclosure. FIG. 8A illustrates handpiece 800 in fully retracted configuration. In various embodiments, handpiece 800 couples to tube 222. In one or more embodiments, handpiece 800 includes inner bore 812, channel 814, and extension control 816 disposed in inner bore 812 and channel 814. In some examples, extension control 816 is configured to actuate within channel 814 in a first direction 830 and in a second direction 840. For example, applying a force vector to a portion of extension control 816 having a direction oriented in the first direction 830 actuates extension control 816 within channel 814 such that a distance between tube 222 and extension control 816 may be increased. Conversely, applying a force vector to a portion of extension control 816 having a direction oriented in the second direction 840 actuates extension control 816 within channel 814 such that the distance between tube 222 and extension control 816 can be decreased.

A functional component in the form of tool 818 of handpiece 800 is disposed in tube 222, inner bore 812, and extension control 816 such that extension control 816 is directly or indirectly fixed to tool 818. Tool 818 is described for purposes of illustration and example, and it is to be appreciated that tool 818 can be replaced with diathermy tip 722, optical fiber 726, soft tip 730, etc. In some embodiments, distal end 820 of tool 818 can include a membrane scraper, such as a flexible loop membrane scraper. In other embodiments, distal end 820 of tool 818 can include a forceps, a scissors, etc. In one or more embodiments, because extension control 816 is fixed to tool 818, an actuation of extension control 816 also actuates tool 818. In a fully retracted configuration as in FIG. 8A, extension control 816 is fully actuated within channel 814 in the first direction 830, and tool 818 is fully retracted relative to tube 222 such that distal end 820 of tool 818 is disposed within tube 222.

FIG. 8B illustrates handpiece 800 in a partially extended configuration. In various examples, application of a force vector to a portion of extension control 816 having a direction oriented in the second direction 840 is configured to actuate extension control 816 within channel 814 in the second direction 840. In one or more embodiments, an actuation of extension control 816 within channel 814 in the second direction 840 is configured to extend tool 818 relative to tube 222 such that distal end 820 of tool 818 extends out from tube 222.

FIG. 8C illustrates handpiece 800 in a fully extended configuration. As shown, in the extended configuration, extension control 816 is fully actuated within channel 814 in the second direction 840 and tool 818 is fully extended relative to tube 222. Distal end 820 of tool 818 fully extends out from tube 222 in the fully extended configuration.

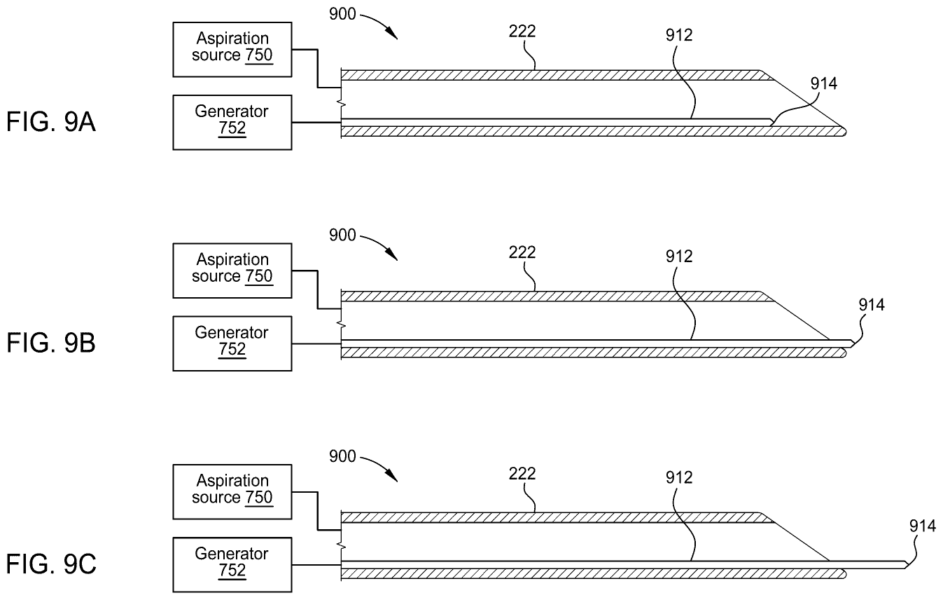

FIGS. 9A-9C illustrate enlarged cross-sectional side views of exemplary configurations of a probe 900 which may be representative of variations of probe 720a with a functional component in the form of an extendable diathermy tip 912, in accordance with certain embodiments of the present disclosure. The probe 900 is illustrated to include (or connect/attach to) the aspiration source 750 and the generator 752. FIG. 9A illustrates the probe 900 in a fully retracted configuration. When the handpiece of probe 900 is in a fully retracted configuration, distal end 914 of diathermy tip 912 is disposed within tube 222.

FIG. 9B illustrates the probe 900 in a partially extended configuration. In one or more embodiments, when the handpiece of probe 900 is in a partially extended configuration, distal end 914 of diathermy tip 912 extends out from tube 222.

FIG. 9C illustrates the probe 900 in a fully extended configuration. In some embodiments, when the handpiece of probe 900 is in a fully extended configuration, distal end 914 of diathermy tip 912 fully extends out from tube 222.

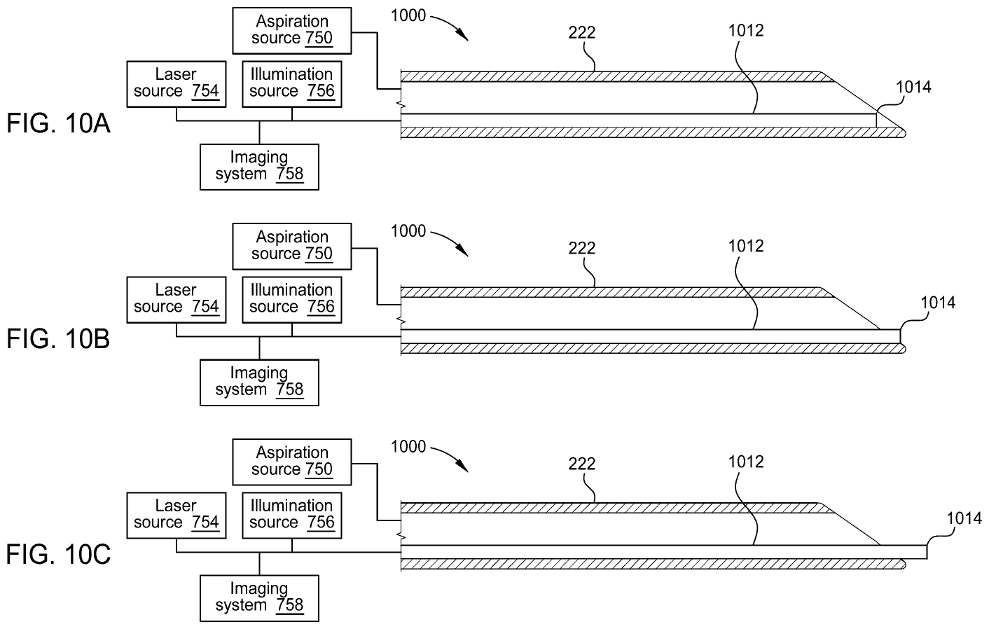

FIGS. 10A-10C illustrate enlarged cross-sectional side views of exemplary configurations of a probe 1000 which may be representative of variations of probe 720b with a functional component in the form of an extendable optic fiber 1012, in accordance with certain embodiments of the present disclosure. The probe 1000 is illustrated to include (or connect/attach to) the aspiration source 750, the laser source 754, the illumination source 756, and the imaging system 758. FIG. 10A illustrates the probe 1000 in a fully retracted configuration. When the handpiece of probe 1000 is in a fully retracted configuration, distal end 1014 of optical fiber 1012 is disposed within tube 222. In the illustrated example, the optical fiber 1012 comprises a straight optical fiber.

FIG. 10B illustrates the probe 1000 in a partially extended configuration. In various embodiments, when the handpiece of probe 1000 is in a partially extended configuration, distal end 1014 of optical fiber 1012 extends out from tube 222.

FIG. 10C illustrates the probe 1000 in a fully extended configuration. In one or more embodiments, when the handpiece of probe 1000 is in a fully extended configuration, distal end 1014 of optical fiber 1012 fully extend out from tube 222.

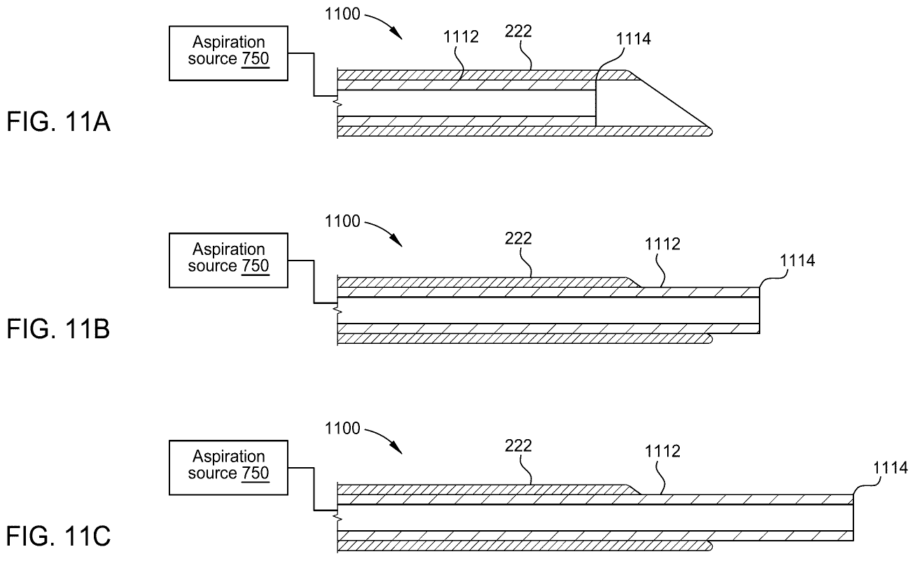

FIGS. 11A-11C illustrate enlarged cross-sectional side views of exemplary configurations of a probe 1100 which may be representative of variations of probe 720c with a functional component in the form of an extendible soft tip 1112, in accordance with certain embodiments of the present disclosure. The probe 1100 is illustrated to include (or connect/attach to) the aspiration source 750. FIG. 11A illustrates the probe 1100 in a fully retracted configuration. As shown, when the handpiece of probe 1100 is in a fully retracted configuration, distal end 1114 of soft tip 1112 is disposed within tube 222. In one or more embodiments, when distal end 1114 of soft tip 1112 is disposed within tube 222, the probe 1100 can be inserted through a valved cannula such as trocar cannulas 150 without bending or deforming soft tip 1112.

FIG. 11B illustrates the probe 1100 in a partially extended configuration. In various embodiments, when the handpiece of probe 1100 is in a partially extended configuration, distal end 1114 of soft tip 1112 extends out from tube 222. For example, after inserting the probe 1100 though the valved cannula, distal end 1114 of soft tip 1112 is extended out from tube 222 for manipulating a portion of retina 104.

FIG. 11C illustrates the probe 1100 in a fully extended configuration. In some embodiments, when the handpiece of probe 1100 is in the fully extended configuration, distal end 1114 of soft tip 1112 fully extends out from tube 222.

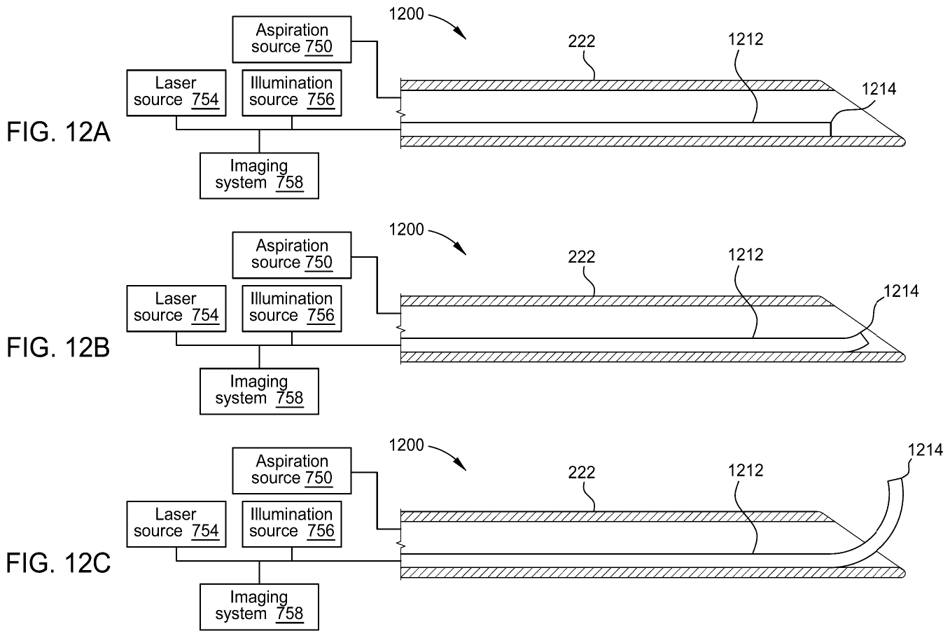

FIGS. 12A-12C illustrate enlarged cross-sectional side views of exemplary configurations of a probe 1200 with a functional component in the form of an extendable and bendable optical fiber 1212, in accordance with certain embodiments of the present disclosure. The probe 1200 is illustrated to include (or connect/attach to) the aspiration source 750, the laser source 754, the illumination source 756, and the imaging system 758. FIG. 12A illustrates the probe 1200 in a fully retracted configuration. In various embodiments, the probe 1200 is an adjustably curved endoilluminator, an adjustably curved laser probe, an adjustably curved illuminated laser probe, an adjustably curved endoscope, etc. Notably, in one or more embodiments, optical fiber 1212 can include multiple optical fibers 1212 which can have the same functionality or different functionalities as described with respect to FIG. 7B. For example, optical fiber 1212 may be configured as an endoscopic camera.

In some examples, when the handpiece of probe 1200 is in a fully retracted configuration, distal end 1214 of optical fiber 1212 is disposed within tube 222. In one or more embodiments, a portion of optical fiber 1212 is enclosed in (e.g., disposed in a sleeve of) a shape memory material (e.g., nitinol) which has a pre-formed curve (e.g., a curve of about 90°). As shown, while the pre-formed curve of the shape memory material is disposed within tube 222, the pre-formed curve is generally straightened and the portion of optical fiber 1212 which is enclosed in the shape memory material is also generally straightened.

FIG. 12B illustrates the probe 1200 in a partially extended configuration. In various embodiments, when the handpiece of probe 1200 is in a partially extended configuration, distal end 1214 of optical fiber 1212 begins to extend out from tube 222, for example, distal end 1214 of optical fiber 1212 begins to extend out from port 228. As illustrated in FIG. 12B, as optical fiber 1212 extends out from tube 222, the pre-formed curve of the shape memory material also begins to extend out from tube 222. Notably, in some examples, as the pre-formed curve of the shape memory material extends out from tube 222, the generally straightened shape memory material begins to curve as the pre-formed curve is no longer generally straightened by tube 222. In one or more embodiments, as the shape memory material curves, optical fiber 1212 also curves.

FIG. 12C illustrates the probe 1200 in a fully extended configuration. In some embodiments, when the handpiece of probe 1200 is in a fully extended configuration, optical fiber 1212 and the shape memory material are fully extended relative to tube 222 such that distal end 1214 of optical fiber 1212 is curved at an angle corresponding to the pre-formed curve of the curve of the shape memory material. For example, as the pre-formed curve of the shape memory material is fully extended from tube 222, the shape memory material curves to the pre-formed curve which also curves optical fiber 1212 to the pre-formed curve. As shown in FIG. 12C, when the handpiece of probe 1200 is in a fully extended configuration, distal end 1214 of optical fiber 1212 is curved about 90° relative to tube 222.

Although the examples described with respect to FIGS. 12A-12C include the shape memory material having the pre-formed curve for curving optical fiber 1212, it is to be appreciated that, in some embodiments, optical fiber 1212 is curved by curving tube 222. In various examples, a portion of tube 222 can include a plurality of apertures such as slits or slots. An application of force to the portion of tube 222 having the plurality of apertures (e.g., by a pull wire or cable) compresses the portion of tube 222 causing tube 222 and optical fiber 1212 to curve. Removing the force applied to the portion of tube 222 decompresses the portion of tube 222 causing tube 222 and optical fiber 1212 to straighten. It is also to be appreciated that examples of curving/bending the optical fiber 1212 are not necessarily limited to optical fibers and may be applied to, for example, the soft tip 1112, the diathermy tip 912, etc.

FIG. 13 illustrates an example surgical system 1300, in accordance with certain embodiments of the present disclosure. The surgical system 1300 is illustrated to include a surgical console 1302 which includes a display 1304, an input device 1306, and a handpiece 1308. In certain embodiments, the handpiece 1308 is representative of handpiece 260 and/or handpiece 800. In some embodiments, the surgical console 1302 includes the aspiration source 750, the generator 752, the laser source 754, the illumination source 756, and/or the imaging system 758. In some other embodiments, the handpiece 1308 includes the aspiration source 750, the generator 752, the laser source 754, the illumination source 756, and/or the imaging system 758. In various examples, the display 1304 is capable of displaying images captured by probe 720b in examples in which probe 720b includes the camera, the lens, the image sensor, etc. In other examples, the surgical system 1300 includes an additional display, separate from the surgical console 1302, configured to display images captured by probe 720b in example which the probe 720b includes the camera, the lens, the image sensor, etc. In one or more examples, a user may interact with the input device 1306 to selectively energize the generator 752, increase or decrease suction from the aspiration source 750, selectively energize the laser source 754, etc.

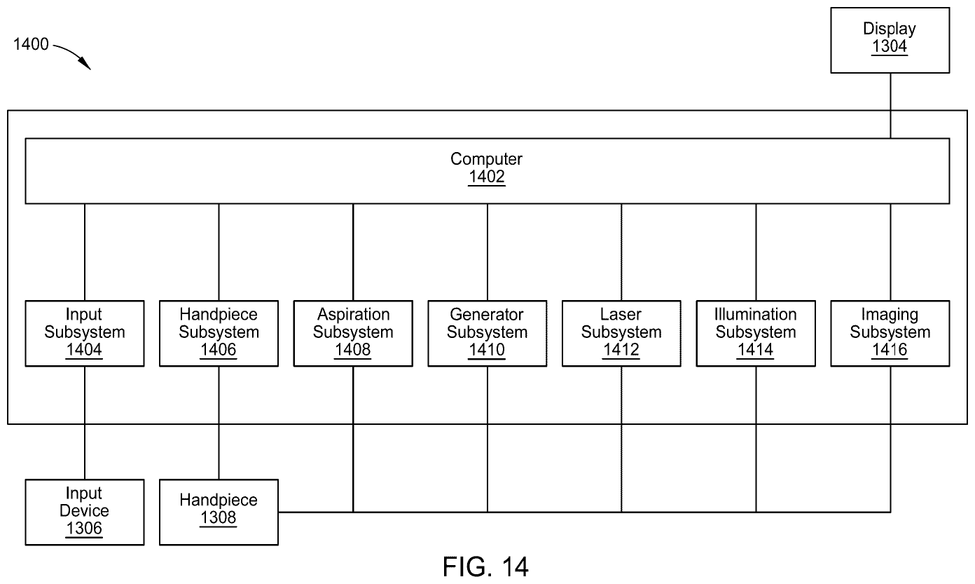

FIG. 14 illustrates subsystems 1400 of the surgical console 1302 of the surgical system of FIG. 13, in accordance with certain embodiments of the present disclosure. Computer 1402 includes memory and a least one processor. The at least one processor executes instructions that cause the at least one processor to generate user interface elements for display by display 1304, respond to inputs received via the input device 1306, etc. Computer 1402 is illustrated as being communicatively coupled by a wired or wireless connection to the input device 1306 via input subsystem 1404 and to the handpiece 1308 via handpiece subsystem 1406. As shown in FIG. 14, computer 1402 may be communicatively coupled by a wired or wireless connection to the handpiece 1308 via aspiration subsystem 1408, generator subsystem 1410, laser subsystem 1412, illumination subsystem 1414, and/or imaging subsystem 1416.

In certain embodiments, the aspiration source 750 is included in the aspiration subsystem 1408. In certain other embodiments, the aspiration source 750 is included in handpiece 260 and/or handpiece 800. In one or more embodiments, the generator 752 is included in the generator subsystem 1410. In one or more other embodiments, the generator 752 is included in handpiece 260 and/or handpiece 800. In various embodiments, the laser source 754 is included in the laser subsystem 1412. In various other embodiments, the laser source 754 is included in handpiece 260 and/or handpiece 800.

In some embodiments, the illumination source 756 is included in the illumination subsystem 1414. In other embodiments, the illumination source 756 is included in handpiece 260 and/or handpiece 800. In certain embodiments, the imaging system 758 is included in the imaging subsystem 1416. In certain other embodiments, the imaging system 758 is included in handpiece 260 and/or handpiece 800.

In summary, embodiments of the present disclosure generally relate to surgical probes for ophthalmic procedures. In particular, the embodiments herein provide probes designed for effective engagement and manipulation of the vitreous and other materials. Such probes include beveled probe tips that increase the area of vacuum generation at a target site without increasing probe gauge, thus facilitating improved tissue engagement as compared to other devices of similar gauge. In certain embodiments, the probes described herein further include one or more texturized surfaces for improved “grabbing” and manipulation of target tissues, and/or one or more surfaces formed of polymeric materials to reduce unwanted damage and enhance the safety thereof. Accordingly, the devices described herein address the deficiencies of certain existing methods and designs, and further reduce the risk of unwanted damage to peripheral tissues.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A surgical probe comprising:
a handpiece configured to be held by a user; and
a tube comprising:
a proximal end coupled to the handpiece;
a distal end opposite the proximal end and comprising a distal tip, wherein the distal tip is beveled and comprises an end face at least partially defining a port for aspiration; and
at least one functional component disposed in the tube.

2. The surgical probe of claim 1, wherein the at least one functional component includes a soft tip extending out from the distal end.

3. The surgical probe of claim 1, wherein the at least one functional component includes a diathermy tip extending out from the distal end, a proximal end of the diathermy tip connected to a generator.

4. The surgical probe of claim 1, wherein the at least one functional component includes an optical fiber, a proximal end of the optical fiber connected to a light source.

5. The surgical probe of claim 4, wherein the optical fiber is configured to transmit light for illumination.

6. The surgical probe of claim 4, wherein the optical fiber is configured to transmit laser light for photocoagulation.

7. The surgical probe of claim 4, further comprising at least one of a lens, a camera, or an image sensor.

8. The surgical probe of claim 1, wherein the at least one functional component is extendable relative to the tube.

9. The surgical probe of claim 8, wherein the at least one functional component includes a soft tip.

10. The surgical probe of claim 8, wherein the at least one functional component includes a diathermy tip.

11. The surgical probe of claim 8, wherein the at least one functional component includes an optical fiber.

12. The surgical probe of claim 11, wherein the optical fiber comprises a straight optical fiber.

13. The surgical probe of claim 11, wherein a portion of the optical fiber is disposed in a shape memory material having a pre-formed curve.

14. The surgical probe of claim 11, wherein the optical fiber is configured to transmit light for illumination.

15. The surgical probe of claim 11, wherein the optical fiber is configured to transmit laser light for photocoagulation.

16. The surgical probe of claim 1, wherein the end face is disposed at an angle between about 10° and about 30° relative to a normal of the longitudinal axis.
